(19) United States
(12) Patent Application Publication
(10) Pub. No.: US 2018/0247705 A1
(43) Pub. Date: Aug. 30, 2018

(54) INFORMATION PROCESSING APPARATUS, INFORMATION PROCESSING METHOD, AND PROGRAM

(71) Applicant: Sony Corporation, Tokyo (JP)

(72) Inventors: Seijiro Inaba, Kanagawa (JP); Hiroshi Ikeda, Tokyo (JP); Nobuho Ikeda, Kanagawa (JP)

(21) Appl. No.: 15/754,699

(22) PCT Filed: Jul. 25, 2016
(86) PCT No.: PCT/JP2016/003450

§ 371 Date: Feb. 23, 2018

(30) Foreign Application Priority Data
Sep. 29, 2015 (JP) 2015-191436

(51) Int. Cl. G16H 20/30

(52) U.S. Cl. CPC G16H 20/30

(57) ABSTRACT

There is provided an information processing method including comparing user information to a plurality of data sets, each data set of the plurality of data sets has candidate information including a candidate model of a motion type for a user, and selecting one candidate model of one data set of the plurality of data sets as a selected model of the motion type for the user based on the comparison of the user information to the plurality of data sets.

### CROSS REFERENCE TO RELATED APPLICATIONS

This application claims the benefit of Japanese Priority Patent Application JP 2015-191436 filed Sep. 29, 2015, the entire contents of which are incorporated herein by reference.

### TECHNICAL FIELD

The present disclosure relates to an information processing apparatus, an information processing method, and a program.

### BACKGROUND ART

Techniques for providing information for guidance that is suitable for the user's individuality, such as a user's body type or the habitual pattern of movement of the user, are developed. An example of the technique for generating information for exercise guidance by performing a calculation using an exercise model that contains a human body model and by analyzing a movement appropriate to the human body model includes a technique disclosed in PTL 1 below.

### CITATION LIST

### Patent Literature

### [PTL 1]

### JP 2013-027629A

### SUMMARY

### Technical Problem

When the technique disclosed in PTL 1 as an example is employed, the analysis of a movement of the human body model using an exercise model stored in a recording medium generates information for exercise guidance. Thus, the information for exercise guidance generated by the technique disclosed in PTL 1 varies in accordance with the exercise model.

However, the exercise model stored in the recording medium is not necessarily an ideal exercise model for every user. Thus, contents indicated by the information for exercise guidance generated on the basis of the previously specified exercise model as in the technique disclosed in PTL 1 as an example are likely not to be an ideal motion for every user.

An embodiment of the present disclosure provides a novel and improved information processing apparatus, information processing method, and program, capable of deciding a model of a motion type allowing the user to perform a motion serving as a model for the user.

### Solution to Problem

According to an embodiment of the present disclosure, there is provided an information processing method including comparing user information to a plurality of data sets, each data set of the plurality of data sets has candidate information including a candidate model of a motion type for a user, and selecting one candidate model of one data set of the plurality of data sets as a selected model of the motion type for the user based on the comparison of the user information to the plurality of data sets.

According to an embodiment of the present disclosure, there is provided an information processing apparatus including an electronic processor, and a memory storing instructions that, when executed by the electronic processor, perform a set of operations including comparing user information to a plurality of data sets, each data set of the plurality of data sets has candidate information including a candidate model of a motion type for a user, and selecting one candidate model of one data set of the plurality of data sets as a selected model of the motion type for the user based on the comparison of the user information to the plurality of data sets.

According to an embodiment of the present disclosure, there is provided an information processing system including a plurality of devices connected to a network, wherein one or more of the plurality of devices is configured to compare user information to a plurality of data sets, each data set of the plurality of data sets has candidate information including a candidate model of a motion type for a user, and select one candidate model of one data set of the plurality of data sets as a selected model of the motion type for the user based on the comparison of the user information to the plurality of data sets.

### Advantageous Effects of Invention

According to the embodiments of the present disclosure, it is possible to decide a model of a motion type allowing the user to perform a motion serving as a model for the user.

Note that the effects described above are not necessarily limited, and along with or instead of the effects, any effect that is desired to be introduced in the present specification or other effects that can be expected from the present specification may be exhibited.

### BRIEF DESCRIPTION OF DRAWINGS

FIG. 1 is a block diagram illustrating an exemplary configuration of an information processing apparatus according to an embodiment of the present disclosure.

FIG. 2 is a diagram illustrated to describe an exemplary hardware configuration of the information processing apparatus according to an embodiment of the present disclosure.

FIG. 3 is a diagram illustrated to describe a process for implementing an information processing method according to an embodiment of the present disclosure.

FIG. 4 is a diagram illustrated to describe a process for implementing an information processing method according to an embodiment of the present disclosure.

FIG. 5 is a flowchart illustrating an example of a process for implementing the information processing method according to an embodiment of the present disclosure.

### DESCRIPTION OF EMBODIMENTS

Hereinafter, (a) preferred embodiment(s) of the present disclosure will be described in detail with reference to the appended drawings. In this specification and the appended drawings, structural elements that have substantially the same function and structure are denoted with the same reference numerals, and repeated explanation of these structural elements is omitted.

The description will be given in the order of items shown below.

1. Information Processing Method according to Present Embodiment
2. Information Processing Apparatus according to Present Embodiment
3. Program according to Present Embodiment

### (Information Processing Method According to Present Embodiment)

First, the configuration of an information processing apparatus according to an embodiment of the present disclosure will be described. As an example, the case will be described where a process for implementing the information processing method according to the present embodiment is performed by the information processing apparatus according to the embodiment.

### <1> Existing Methods

The description is given by exemplifying a case where the user practices sports such as golf or tennis.

Although the following description is given by exemplifying a case where the user is practicing sports, this is similarly applied to the case where the user practices any other actions involving a motion type, such as strength training, cooking, and procedure in medical practice. The motion type according to the present embodiment herein includes one or both of a posture (a pose during an exercise or the like) and a movement.

In practicing a sport, it is sometimes expected that “a coach observes a posture or the like of a user who performs practice and gives guidance to the user on how to improve”. The user who is practicing repeatedly performs the practice to correct the posture or the like by considering a point to be improved which is guided from the coach, thereby achieving skill improvement.

The user who practices sports or the like may sometimes practice voluntarily without receiving the coach's guidance. In this case, the user who performs practice is aiming at achieving skill improvement through repeated practice to be closer to a posture in which user imagines, for example, by “reading a book acting as a coach to check the user's own posture”.

In this connection, the ideal motion for the user who performs practice is more likely to be different for each user because of features in the body of each user. Thus, the ideal motion that is thought by a coach or the ideal motion that is disclosed in the book acting as a coach is not necessarily taken as an ideal motion for the user who performs practice. Furthermore, as described above, even when the information for exercise guidance is generated on the basis of a previously specified exercise model as with the technique disclosed in PTL 1 for example, contents indicated by the generated information for exercise guidance are likely not to be an ideal motion for every user.

When a user performs practice by accepting, as a model, a motion type that is not an ideal motion type for the user who performs practice, this is likely not to lead to an achievement of skill improvement for the user who performs practice. Furthermore, in the above case, an excessive load may be imposed to the body, increasing a risk of injury.

### <2> Overview of Information Processing Method According to Present Embodiment

Thus, the information processing apparatus according to the present embodiment decides a person who become a model of a motion type for the user (sometimes referred to as “model of motion type” hereinafter) in consideration of the user's features (decision process). The information processing apparatus according to the present embodiment decides, as a model of motion type, a candidate for model having a feature closer to that of the user among a plurality of candidates for a model of motion type (sometimes referred to as “candidate for model” hereinafter). The “model” according to the present embodiment can be interchangeable with “example” or “sample”.

The user according to the present embodiment herein is a person to be a target of the information processing method according to the present embodiment. The user corresponds to a user who performs practice such as sports described above as an example.

Examples of the candidate for a model according to the present embodiment include a person having predetermined past record and experience (e.g., objective ones) for any target involving a motion such as sports, for example as described below.

Noted that the candidate for a model according to the present embodiment includes, but not limited to, persons described below.
Professional sports players (sometimes referred to simply as “pro” hereinafter)
Professional sports leaders
Coaches in sports (or instructors)
Experienced doctor or medical specialist

Examples of the user's features according to the present embodiment include one or both of a feature of a user's body and a feature of a user's motion.

In the decision process according to the present embodiment, it is possible to decide, as a model of the motion type, a candidate for a model having a feature of the body (e.g., body type, muscular strength, and flexibility) closer to that of the user by considering the feature of the user's body as a feature of the user.

The decision of a candidate for a model having a feature of the body closer to that of the user as the model of the motion type allows the user to take a motion that is more suitable for its own body as a model.

Thus, it may be possible for the user to improve the skill improvement more effectively. Furthermore, it also is possible to further reduce the possibility of imposing an excessive load to the user's body when a motion of the model of the motion type is performed as a practice of the model.

Moreover, in the decision process according to the present embodiment, it is possible to decide, as a model of the motion type, a candidate for a model having a feature of the body's movement (may include the movement of a tool involving the movement of the body) closer to that of the user by considering the feature of the user's motion as a feature of the user.

The candidate for a model having a feature of the motion close to that of the user is decided as the model of the motion type, and thus a difference in motion between a motion serving as a model and the user's motion is smaller.

Thus, the point to be improved of the motion in the user becomes less, and it may be possible for the user to achieve skill improvement more efficiently. Furthermore, it is possible to further reduce the possibility of imposing an excessive load to the user's body when a motion of the model of the motion type is performed as a practice of the model.

In the decision process according to the present embodiment, as described above, a model of the motion type is decided by considering one or both of the feature of the user's body and the feature of the user's motion as the feature of the user.

Thus, the information processing apparatus according to the present embodiment performs the decision process described above as the process for implementing the information processing method according to the present embodiment, and thus it is possible to decide a model of the motion type that allows the user to perform a motion serving as the model.

### <3> Other Examples of Process for Implementing Information Processing Method According to Present Embodiment

Note that the process for implementing the information processing method according to the present embodiment is not limited to the decision process described above.

For example, the information processing apparatus according to the present embodiment is capable of further performing one or more processes using a result obtained by the decision process. Examples of the process using a result obtained by the decision process include one or both of an analysis process and a notification process, which will be described later.

Note that “the decision process” and “the decision process and one or both of the analysis process and the notification process” are those obtained by dividing the process for implementing the information processing method according to the present embodiment for convenience sake. Thus, in the process for implementing the information processing method according to the present embodiment, for example, “the decision process and one or both of the analysis process and the notification process” can be understood as one process. In addition, in the process for implementing the information processing method according to the present embodiment, each of “the decision process” and “the decision process and one or both of the analysis process and the notification process” can also be understood as two or more processes (using any suitable division way).

### (Information Processing Apparatus According to Present Embodiment)

An exemplary configuration of the information processing apparatus according to the present embodiment capable of performing the process for implementing the information processing method according to the present embodiment described above will be described, and the process for implementing the information processing method according to the present embodiment will be described in detail.

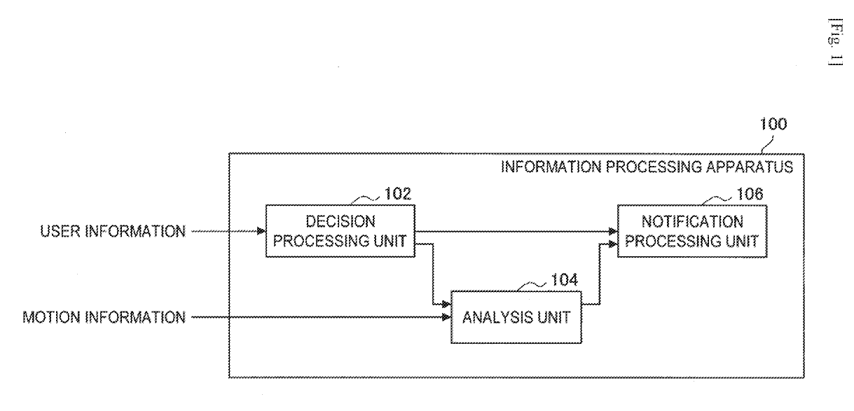

FIG. 1 is a block diagram illustrating an exemplary configuration of an information processing apparatus 100 according to the present embodiment. The information processing apparatus 100 is configured to include a decision processing unit 102, an analysis unit 104, and a notification processing unit 106, as an example.

The information processing apparatus 100 may be configured to include a controller (not shown), read-only memory (ROM, not shown), random access memory (RAM, not shown), a storage unit (not shown), a communication unit (not shown), an operation unit (not shown) operable by a user, a display unit (not shown) for displaying various pictures on a display screen, and so on. In the information processing apparatus 100, the components described above are interconnected via a bus that serves as a data transmission channel.

The controller (not shown) is configured to include one or more processors constituted by an arithmetic logic circuit such as micro processing unit (MPU) and various processing circuits, and controls the entire information processing apparatus 100. The controller (not shown) may function as one or more of the decision processing unit 102, the analysis unit 104, and the notification processing unit 106 in the information processing apparatus 100.

One or more of the decision processing unit 102, the analysis unit 104, and the notification processing unit 106 may be configured as a dedicated (or general purpose) circuit (e.g., a separate processor from the controller (not shown)), which is capable of performing the process of each of the decision processing unit 102, the analysis unit 104, and the notification processing unit 106.

The ROM (not shown) is used to store data for control such as programs and operation parameters used by the controller (not shown). The RAM (not shown) is used to temporarily store programs and other instructions for execution by the controller (not shown).

The storage unit (not shown) is a storage mechanism provided in the information processing apparatus 100, and stores data, for example, candidate information (described later) used in the information processing method according to the embodiment or stores various data such as a variety of applications.

Examples of the storage unit (not shown) include a magnetic recording medium such as hard disk, and nonvolatile memory such as flash memory. The storage unit (not shown) may be removable from the information processing apparatus 100.

Examples of the communication unit (not shown) include a communication interface described later. Examples of the operation unit (not shown) include an operation input device described later. Examples of the display unit (not shown) include a display device described later.

### (Exemplary Hardware Configuration of Information Processing Apparatus 100)

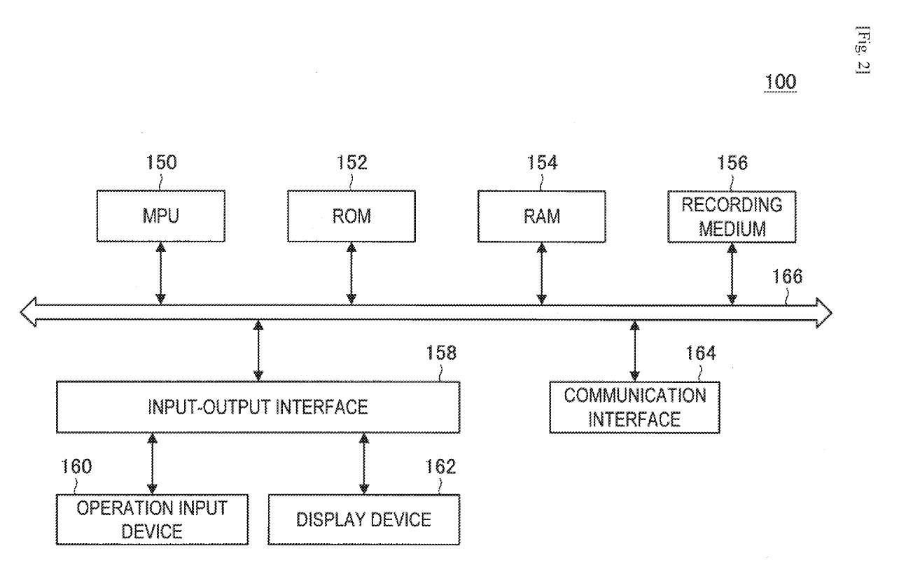

FIG. 2 is a diagram illustrated to describe an exemplary hardware configuration of the information processing apparatus 100 according to the present embodiment. The information processing apparatus 100 may be configured to include an MPU 150, a ROM 152, a RAM 154, a recording medium 156, an input-output interface 158, an operation input device 160, a display device 162, a communication interface 164. In the information processing apparatus 100, the components are interconnected via a bus 166 that serves as a data transmission channel.

The MPU 150 may be configured to include one or more processors constituted by an arithmetic logic circuit such as MPU and various processing circuits, and functions as the controller (not shown) that controls the entire information processing apparatus 100. The MPU 150 serves as the decision processing unit 102, the analysis unit 104 or the notification processing unit 106 in the information processing apparatus 100. One or more of the decision processing unit 102, the analysis unit 104, and the notification processing unit 106 may be configured as a dedicated (or general purpose) circuit (e.g., a separate processor from the MPU 150) capable of performing the process of each of the decision processing unit 102, the analysis unit 104, and the notification processing unit 106.

The ROM 152 stores data for control, such as programs and operation parameters used by the MPU 150. The RAM 154 stores temporarily programs and other data executed by the MPU 150.

The recording medium 156 functions as the storage unit (not shown), and stores data relating to the information processing method according to the embodiment such as candidate information (described later) and a variety of data including various types of applications. Examples of the recording medium 156 include a magnetic recording medium such as hard disk, and nonvolatile memory such as flash memory. The recording medium 156 may be removable from the information processing apparatus 100.

The input-output interface 158 is used for connection of the operation input device 160 and the display device 162. The operation input device 160 functions as the operation unit (not shown). The display device 162 functions as the display unit (not shown). Examples of the input-output interface 158 include a universal serial bus (USB) terminal, a digital visual interface (DVI) terminal, a high-definition multimedia interface (HDMI, registered trademark) terminal, and various types of processing circuits.

The operation input device 160 is provided, for example, on the information processing apparatus 100 and is connected to the input-output interface 158 within the information processing apparatus 100. Examples of the operation input device 160 include a button, a direction key, a rotation type selector such as a jog dial, and a combination thereof.

The display device 162 is provided, for example, on the information processing apparatus 100 and is connected to the input-output interface 158 within the information processing apparatus 100. Examples of the display device 162 include a liquid crystal display (LCD) and an organic electro-luminescence (EL) display (or also referred to as an organic light emitting diode (OLED) display).

It will be understood that the input-output interface 158 may be connected to an external device, such as an external operation input device (e.g., keyboard or mouse) or an external display device of the information processing apparatus 100. The display device 162 may be a device such as a touch panel on which a display process and the user's operation can be performed.

The communication interface 164 is a communication mechanism that is provided in the information processing apparatus 100. The communication interface 164 functions as a communication unit (not shown) for communicating with “an external device such as a sensor used to detect a motion of a user” or “an external apparatus such as one or more servers used to store one or both of user information (described later) and motion information (described later)”, by wire or wireless over a network (or directly).

Examples of the communication interface 164 include a communication antenna and radio frequency (RF) circuit (wireless communication), an IEEE 802.15.1 port and transmission-reception circuit (wireless communication), an IEEE 802.11 port and transmission-reception circuit (wireless communication), or a local area network (LAN) terminal and transmission-reception circuit (wired communication).

The information processing apparatus 100 having, for example, the configuration shown in FIG. 2 performs the process for implementing the information processing method according to the embodiment. The hardware configuration of the information processing apparatus 100 according to the embodiment is not limited to that shown in FIG. 2.

For example, the information processing apparatus 100, when it communicates with an external apparatus through an external communication device connected thereto or when it performs a process as a stand-alone device, may have a configuration that does not include the communication interface 164. The communication interface 164 may have a configuration capable of communicating with one or more external apparatuses using a plurality of communication schemes.

As one example, the information processing apparatus 100 may be configured to further include one or both of a sensor used to acquire user information (described later) and a sensor used to acquire motion information (described later).

As one example, the information processing apparatus 100 can have a configuration that does not include one or more of the recording medium 156, the operation input device 160, and the display device 162.

As one example, some or all of the components illustrated in FIG. 2 (or components according to a modified example) may be implemented by one or more integrated circuits (ICs).

Referring back to FIG. 1, an exemplary configuration of the information processing apparatus 100 will be described.

### <1> Decision Processing Unit 102

The decision processing unit 102 plays a leading role in performing the decision process described above. The decision processing unit 102 decides a model of the motion type for the user corresponding to user information on the basis of the user information and a plurality pieces of candidate information which each indicates a candidate for a model of the motion type.

The user information according to the present embodiment herein is data relating to the user. Examples of the user information include one or both of data indicating a feature of the user's body and data indicating a result obtained from the user's motion.

Examples of data indicating a feature of the user's body as an example of the user information include one or more values each indicating a feature of the user's body. The value indicating a feature of the user's body is referred to as “feature value” hereinafter.

Furthermore, examples of the feature of the user's body indicated by the data indicating a feature of the user's body include one or a combination of two or more of examples described below. The feature value of the user indicated by the user information according to the present embodiment is not limited to feature values corresponding to features described below. For example, one or both of a feature relating to the length and a feature relating to the weight of the body described below may be represented by a single index like a feature relating to the body type. Furthermore, the feature value of the user indicated by the user information according to the present embodiment may be one or more any index values that can indicate a feature of the body.
Feature relating to the length of a body: height, arm's length, leg's length, size of hand, size of foot, or the like
Feature relating to the weight: body weight, condition of flesh on each part (e.g., shoulder, arm, chest, abdomen, back, buttocks, and leg) of body
Feature relating to muscular strength for each part: grip strength, back muscle strength, abdominal muscle strength, or the like
Feature relating to flexibility of each joint or muscle: flexibility of hip joint, shoulder joint, bending forward, trunk lift test, or the like

The feature value of the feature relating to the length of a body may be one obtained by measurement using a measure or may be one obtained by estimation from a captured image obtained by photographing the user.

The feature value of the feature relating to the weight of a body may be one obtained by measurement using a weight scale or may be one obtained by measurement using a measuring instrument such as magnetic resonance imaging (MRI).

The feature value of the feature relating to muscular strength for each part may be obtained, for example, by measurement using a dedicated measuring instrument or strength training equipment.

The feature value of the feature relating to flexibility of each joint or muscle may be obtained, for example, by measurement of flexibility through the calisthenics performed by the user. For example, the feature value of the feature relating to flexibility of each joint or muscle is represented by values ranging from 0.0 to 1.0 by normalizing a measured value using a predetermined maximum movable range for each part. Furthermore, the feature value of the feature relating to flexibility of each joint or muscle may be an evaluation value of the qualitative evaluation of a plurality of stages, like five-stage evaluation in the motion with both arms folded on the back as described below.
Stage 5 (very good): case where fingers of both arms are crossed, as an example
Stage 4 (reasonably good): case where fingertips are touching each other, as an example
Stage 3 (medium): case where distance between fingertips of both arms is within 5 cm, as an example
Stage 2 (bad): case where distance between fingertips of both arms is within 10 cm, as an example
Stage 1 (very bad): case where distance between fingertips of both arms is greater than 10 cm, as an example

When the feature of the user's body is measured as described above, the user information indicating the feature of the user's body is generated by allowing a result as data obtained by measurement to be entered manually or automatically. When the feature of the user's body is estimated from a captured image as described above, the user information indicating the feature of the user's body is generated in a device performing a process on the estimation. The process on the estimation of the feature of the user's body based on the captured image herein may be performed by the information processing apparatus 100 or may be performed by an external apparatus of the information processing apparatus 100.

Examples of the data indicating a result obtained from the user's motion as an example of the user information include “data indicating a result obtained by detection of one or both of the movement of the user's body and the movement of a tool or the like used by the user (sometimes referred to as “movement detection data” hereinafter) or “data that can be used to estimate one or both of the movement of the user's body and the movement of a tool such as a hitting tool used by the user (sometimes referred to as “movement estimation data” hereinafter)”.

Examples of the movement detection data as an example of the motion information include data indicating a detection result obtained from detection by any movement detection sensor capable of detecting the movement of a detection target, such as optical movement detection sensors (marker-based or markerless-based sensors), magnetic movement detection sensors, and inertial-based movement detection sensors. The user's movement is detected using the movement detection sensors as described above, and thus the user information can indicate one or both of the three-dimensional movement of the user's joint and the three-dimensional movement of a tool such as hitting tools.

Examples of the movement estimation data include any data that can be used to estimate the movement of a detection target, such as a captured image of a user that is photographed. For example, the process on the estimation of the user's movement or the like based on the captured image (an example of the movement estimation data) herein may be performed by the information processing apparatus 100 or may be performed by an external apparatus of the information processing apparatus 100.

The decision processing unit 102 uses one or both of the data indicating a feature of the user's body and the data indicating a result obtained from the user's motion for example as described above as the user information in the decision process.

The user information according to the present embodiment is not limited to one or both of the data indicating a feature of the user's body and the data indicating a result obtained from the user's motion. For example, the user information may further contain one or both of sex of the user and age of the user.

The candidate information according to the present embodiment is data relating to a candidate for a model. Examples of the candidate information include one or both of data indicating a feature of the body of a candidate for the model and data indicating a result obtained from the motion of a candidate for the model.

Examples of the data indicating a feature of the user's body as an example of the candidate information include data that is similar to the data indicating a feature of the user's body.

Examples of the data indicating a result obtained from the motion of a candidate for the model as an example of the candidate information include data that is similar to the data indicating a result obtained from the user's motion.

The candidate information according to the present embodiment is not limited to one or both of the data indicating a feature of the body of a candidate for the model and the data indicating a result obtained from the motion of a candidate for the model. For example, the candidate information according to the present embodiment may further contain one or both of the sex of a candidate for the model and the age of a candidate for the model.

The decision processing unit 102 decides a model of the motion type for the user from among candidates for the model corresponding to the candidate information on the basis of the user information and the candidate information as described above in the decision process.

More specifically, the decision processing unit 102 decides a model of the motion type, for example by performing any one of a process according to a first example shown in item (1) below to a process according to a fourth example shown in item (4) below.

(1) First Example of Decision Process: Case where User Information and Candidate Information Each Indicates a Feature of the Body

When the user information and the candidate information each indicates a feature of the body, the decision processing unit 102 decides, as a model of the motion type, a candidate for the model having a feature of the body that is closer to that of the user.

For example, the user information may indicate one or more feature values indicating the feature of the user's body and the candidate information may indicate one or more feature values indicating the feature of the body of the candidate for the model. In this case, the decision processing unit 102 decides a model of the motion type by performing an arithmetic operation using these feature values.

Specifically, the decision processing unit 102 calculates a “sum total value of the differences between respective feature values in the user information and the candidate information” as shown Formulas (1) to (3) below. The “sum total value of the differences between respective feature values in the user information and the candidate information” according to the present embodiment is sometimes referred to as “sum total value” or “difference sum total value” hereinafter.

$$\begin{matrix}[ Math . 1 ] & \\ {diff}_{l} = {\sum }_{i = 1}^{n} {w}_{{l}_{i}} \cdot \vert {l}_{i} - {l}_{i}^{′} \vert & Formula ( 1 ) \\ [ Math . 2 ] & \\ {diff}_{p} = {\sum }_{i = 1}^{n} {w}_{{p}_{i}} \cdot \vert {p}_{i} - {p}_{i}^{′} \vert & Formula ( 2 ) \\ [ Math . 3 ] & \\ {diff}_{f} = {\sum }_{i = 1}^{n} {w}_{{f}_{i}} \cdot \vert {f}_{i} - {f}_{i}^{′} \vert & Formula ( 3 )\end{matrix}$$

In Formula (1), “diffₗ” denotes a sum total value based on a feature value corresponding to the feature relating to the length of a body.

In Formula (1), “lᵢ” is a feature value of the feature relating to the length of the user's body. In Formula (1), “l′ₜ” is a feature value of the feature relating to the length of the body corresponding to the “lᵢ” in a candidate of a model. In Formula (1), “wₗᵢ” is a weighting coefficient corresponding to each of features relating to the length of the body. The decision processing unit 102 specifies a weighting coefficient corresponding to each of features relating to the length of the body, for example by referring to a table (or database) in which the feature relating to the length of the body and the weighting coefficient are associated with each other. The table in which the feature relating to the length of the body and the weighting coefficient are associated with each other may be one prepared for each motion of the user, such as every sport. The decision processing unit 102 specifies a weighting coefficient corresponding to each of the features relating to the length of the body by referring to a table corresponding to a motion performed by the user among tables stored in a recording medium such as a storage unit (not shown).

In Formula (2), “diffₚ” denotes the sum total value based on the feature value corresponding to the feature relating to the muscular strength of each part.

In Formula (2), “pᵢ” is the feature value of the feature relating to the muscular strength of each part in the user. In Formula (2), “p′ᵢ” is the feature value of the feature relating to the muscular strength of each part corresponding to “pᵢ” in a candidate of the model. In Formula (2), “wₚᵢ” is the weighting coefficient corresponding to each of features relating to the muscular strength of each part. The decision processing unit 102 specifies a weighting coefficient corresponding to each of features relating to the muscular strength of each part, for example by referring to a table (or database) in which the feature relating to the muscular strength of each part and the weighting coefficient are associated with each other. The table in which the feature relating to the muscular strength of each part and the weighting coefficient are associated with each other may be one prepared for each motion of the user, such as every sport.

In Formula (3), “diff_{f}” denotes the sum total value based on the feature value corresponding to the feature relating to flexibility of each joint or muscle.

In Formula (3), “fᵢ” is the feature value of the feature relating to the flexibility of each joint or muscle in the user. In Formula (3), “f′ᵢ” is the feature value of the feature relating to the flexibility of each joint or muscle corresponding to “fᵢ” in a candidate of the model. In Formula (3), “w_{fi}” is the weighting coefficient corresponding to each of features relating to the flexibility of each joint or muscle. The decision processing unit 102 specifies a weighting coefficient corresponding to each of features relating to the flexibility of each joint or muscle, for example by referring to a table (or database) in which the feature relating to the flexibility of each joint or muscle and the weighting coefficient are associated with each other. The table in which the feature relating to the flexibility of each joint or muscle and the weighting coefficient are associated with each other may be one prepared for each motion of the user, such as every sport.

A method of calculating the sum total value according to the present embodiment is not limited to the examples shown in Formulas (1) to (3).

For example, the decision processing unit 102 is also capable of calculating the sum total value by normalizing each feature value, as shown in Formula (4) below. Formula (4) below indicates a calculation example in the case where the sum total value is calculated by normalizing the feature value corresponding to the feature relating to the length of the body. Even in other features of the body, it is possible to normalize each feature value to calculate the sum total value, which is similar to Formula (4) below.

$$\begin{matrix}[ Math . 4 ] & \\ \{ \begin{matrix}{diff}_{l} = {\sum }_{i = 1}^{n} {w}_{{\tilde{l}}_{i}} \cdot \vert {\tilde{l}}_{i} - {\tilde{l}}_{i}^{′} \vert \\ {\tilde{l}}_{i} = \frac{{l}_{i}}{{l}_{x}} \\ {\tilde{l}}_{i}^{′} = \frac{{l}_{i}^{′}}{{l}_{x}^{′}}\end{matrix} & Formula ( 4 )\end{matrix}$$

In Formula (4), “lₓ” is the feature value serving as a reference for normalization in the user, and “l′ₓ” is the feature value serving as a reference of normalization in a candidate for the model. The feature value serving as a reference for normalization may be a fixed feature value such as a feature value indicating the height, or may be changed for each feature value to be normalized such as normalization of the length of the forearm by the length of the upper arm or normalization of the length of the lower leg by the length of the thigh.

As described above, the feature of the body indicated by each of the user information and the candidate information can be classified into a plurality of segments, like the feature relating to the length or the feature relating to the weight of the body.

As described above, when the feature of the body indicated by each of the user information and the candidate information is classified into a plurality of segments, the decision processing unit 102 can calculate the sum total value on the basis of the sum value for each segment. As one example, the decision processing unit 102 calculates the sum total value diff on the basis of the sum value for each segment calculated in the above Formulas (1) to (3), as shown in Formula (5) below.

[Math. 5]

diff=a·diffₗ+b·diffₚ+c·diff_{f} Formula (5)

In Formula (5), “a”, “b”, and “c” are weighting coefficients for each segment. The weighting coefficient for each segment is set for each motion performed by the user, such as every sport.

For example, as shown in Formula (5), the decision processing unit 102 is capable of calculating a sum total value that is weighted for each segment. The weighting for each segment allows the difference in magnitude of the sum total value for each segment to be reduced. Note that the decision processing unit 102 is capable of calculating the sum total value without performing the weighting for each segment.

As described above, when the sum total value is calculated by an arithmetic operation using a feature value, the decision processing unit 102 decides a candidate for a model having the smallest sum total value as the model of the motion type.

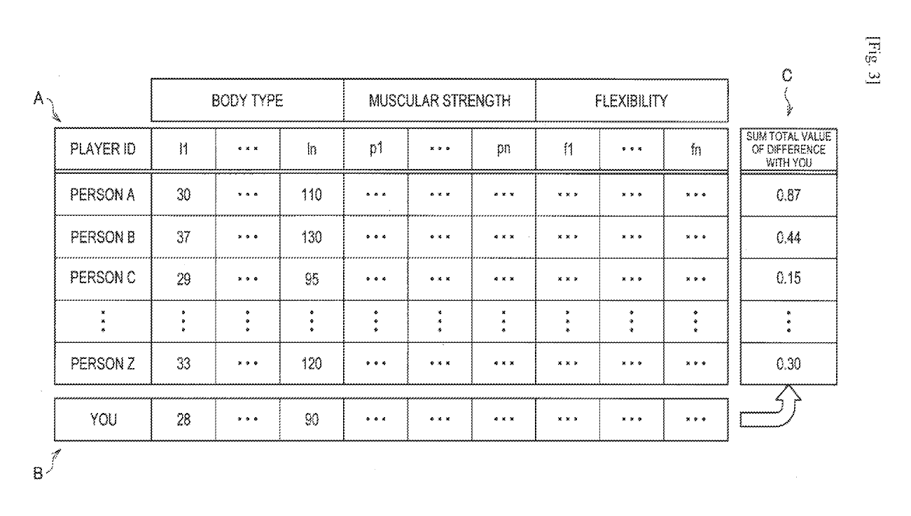

FIG. 3 is a diagram illustrated to describe a process for implementing the information processing method according to the present embodiment, and illustrates an overview of a process in the decision processing unit 102.

The portion A of FIG. 3 illustrates an example of a feature value of a candidate for a model indicated by the candidate information. In the portion A of FIG. 3, “PERSON A”, “PERSON B”, . . . , “PERSON Z” each corresponds to a candidate for a model. The portion B of FIG. 3 illustrates an example of a feature value of the user indicated by the user information. In the portion B of FIG. 3. “YOU” corresponds to the user. In addition, the portion C of FIG. 3 illustrates an example of the sum total value calculated by Formula (5) above. In the portion C of FIG. 3, the sum total value corresponding to “PERSON C” is assumed to have the smallest value.

As one example, when the sum total value shown in the portion C of FIG. 3 is calculated, the decision processing unit 102 decides “PERSON C” that is the candidate for the model having the smallest sum total value as the model of the motion type.

(2) Second Example of Decision Process: Case where User Information and Candidate Information Each Indicates a Result Obtained from a Motion

When the user information and the candidate information each indicates a result obtained from the motion, the decision processing unit 102 decides a candidate for a model having a feature of a motion closer to that of the user as the model of the motion type.

For example, the decision processing unit 102 extracts a feature of the motion from each of the user information and the candidate information. Then, the decision processing unit 102 decides a model of the motion type on the basis of the difference between the extracted features of the motion. The decision processing unit 102 decides a candidate of a model having a smaller difference between the extracted features of the motion as the model of the motion type.

The decision processing unit 102 extracts a feature of the motion, for example by calculating a feature value of the motion from each of the user information and the candidate information. The decision processing unit 102 decides a candidate of a model having the smallest difference between the calculated feature values as the model of the motion type.

As a specific example, when the user information and the candidate information are data of a result obtained by performing motion capture, the decision processing unit 102 calculates a feature amount of the movement from each of the user information and the candidate information. The decision processing unit 102 analyzes main components for each of the user information and the candidate information and sets the coefficient of an eigenvector to be the feature value. The decision processing unit 102 decides a candidate of a model having the smallest difference between the calculated coefficients of the eigenvector as the model of the motion type.

The process of extracting a feature of the motion to decide a model of the motion type is not limited to the example described above.

For example, the decision processing unit 102 is also capable of deciding a model of the model type by extracting a posture of each of the user and the candidate of the model as the feature of the motion on the basis of each of the user information and the candidate information. The decision processing unit 102 decides a candidate for a model having the smallest difference between postures as the model of the motion type.

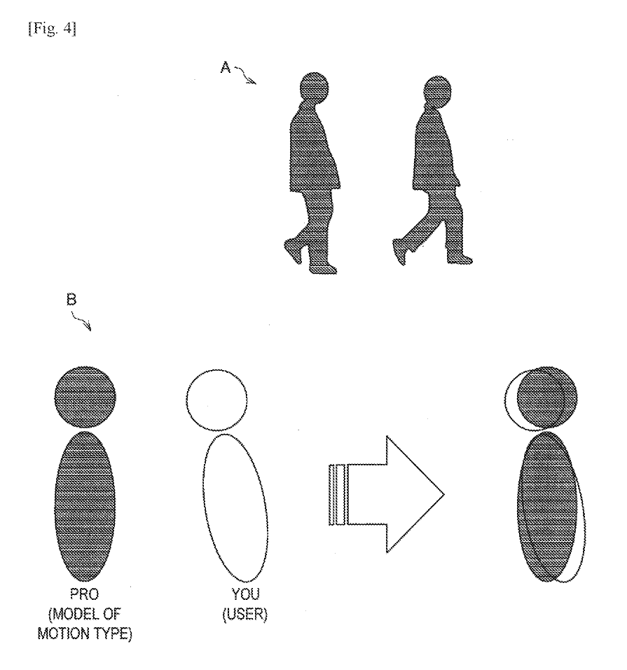

FIG. 4 is a diagram illustrated to describe a process for implementing the information processing method according to the present embodiment, and illustrates an overview of a process performed in the decision processing unit 102.

The decision processing unit 102 generates a silhouette image of some postures during motion in each of the user and the candidate for the model on the basis of each of the user information at a plurality of time points and the candidate information at a plurality of time points, for example as shown in the portion A of FIG. 4. The generation of the silhouette image corresponds to the process for extraction of a feature of the motion.

The decision processing unit 102 causes the silhouette image corresponding to the user that is corresponded to the motion and the silhouette image corresponding to the candidate of the model to be overlapped with each other, for example as shown in the portion B of FIG. 4. The decision processing unit 102 sets a value indicating the overlapping degree (i.e., a value indicating the similarity of a motion) to be the feature value. Examples of the feature value concerning the silhouette image include a value indicating the ratio of the overlapped area to the area of the entire silhouette image corresponding to the user. The feature value concerning the silhouette image is not limited to the example described above, but may be any value that can be used to indicate the overlapping degree.

### (3) Third Example of Decision Process

The decision processing unit 102 is capable of performing a process in which the process according to the first example described in the above item (1) and the process according to the second example described in the above item (2) are combined.

An example of the process of performing the combined processes includes, when the model of the motion type is not narrowed down to one person by one process of the process according to the first example described in the above item (1) and the process according to the second example described in the above item (2), deciding a model of the motion type of one person by the other process. For example, when there are a plurality of candidates for the model having the smallest sum total value by the sum total value concerning the first example described in the above item (1), the decision processing unit 102 decides a model of the motion type by further performing the process according to the second example described in the above item (2) on the basis of the candidate information corresponding to these plurality of candidates for the model.

An example of the process in which the process according to the first example described in the above item (1) and the process according to the second example described in the above item (2) are combined is not limited to the example described above, but may be any process capable of deciding a model of the motion type by combining the process according to the first example described in the above item (1) and the process according to the second example described in the above item (2).

### (4) Fourth Example of Decision Process

The decision process according to the present embodiment is not limited the process according to the first example described in the above item (1) to the process according to the third example described in the above item (3).

As described above, the user information may further indicate the sex of the user. The candidate information may further indicate the sex of a candidate for the model.

As described above, when the user information and the candidate information each indicates the sex of each user, the decision processing unit 102 is capable of deciding a model of the motion type by considering the sex of each of the user and the candidate for the model.

Specifically, the decision processing unit 102 excludes a candidate of the model having the sex different from the user. Then, after the candidate of the model is excluded, the decision processing unit 102 decides a model of the motion type by performing any one of the process according to the first example described in the above item (1) to the process according to the third example described in the above item (3).

For example, the exclusion of the candidate of the model having the sex different from the user by the decision processing unit 102 prevents a candidate of the model having the sex different from the user from being decided as the model of the motion type.

Thus, the user is able to set a motion that is more suitable for its own body to be the model, thereby achieving the skill improvement more effectively. In addition, the ability to set a motion that is more suitable for its own body to be the model allows the possibility of imposing an excessive load to the user's body when a motion of the model of the motion type is performed as a practice of the model to be further reduced.

Furthermore, as described above, the user information may further indicate the age of the user. The candidate information may further indicate the age of a candidate for the model.

As described above, when the user information and the candidate information each indicates the age of each user, the decision processing unit 102 is capable of deciding a model of the motion type in consideration of the age gap between the user and the candidate of the model.

Specifically, the decision processing unit 102 excludes a candidate for the model whose age gap from the user is greater than a predetermined threshold (or a candidate for the model whose age gap from the user is greater than or equal to the predetermined threshold, and this is similarly applied to the following description). Then, after the candidate of the model is excluded, the decision processing unit 102 decides a model of the motion type by performing any one of the process according to the first example described in the above item (1) to the process according to the third example described in the above item (3). The predetermined threshold may be a fixed value that is set in advance, or may be a variable value that can be changed by the user operation by the user or the like of the information processing apparatus 100.

For example, the candidate of the model can be excluded in consideration of the age gap between the user and the candidate for the model by the decision processing unit 102 as described above. Thus, the candidate of the model whose age gap from the user is greater than the predetermined threshold is prevented from being decided as the model of the motion type.

Thus, it is possible for the user to set a motion that is more suitable for its own body to be the model, thereby achieving the skill improvement more effectively. In addition, the ability to set a motion that is more suitable for its own body to be the model allows the possibility of imposing an excessive load to the user's body when a motion of the model of the motion type is performed as a practice of the model to be further reduced.

The decision processing unit 102 is capable of deciding a model of the motion type after the candidate for the model is excluded according to one or both of the sex of each user indicated by each of the user information and the candidate information and the age of each user indicated by each of the user information and the candidate information as described above.

### <II> Analysis Unit 104

The analysis unit 104 plays a role in performing one process using a result obtained by the decision process, and it plays a leading role in the analysis process as the process using the result obtained by the decision process. The analysis unit 104 analyzes the user's motion on the basis of the motion information indicating the user's motion and the motion information corresponding to the decided model of the motion type.

The motion information according to the present embodiment herein is data indicating a result obtained from the user's motion. Examples of the motion information include the movement detection data or the movement estimation data.

When the motion information indicating the user's motion is the movement detection data, the motion information indicating the user's motion is acquired, for example, from the movement detection sensor.

When the motion information indicating the user's motion is the movement estimation data, the motion information indicating the user's motion is acquired from an imaging device for capturing the user. The analysis unit 104 is capable of estimating the user's movement by performing a process for estimating any movement on the captured image (an example of the movement estimation data) acquired from the imaging device.

When the user information contains the data indicating a result obtained from the user's motion, the analysis unit 104 is capable of using the data indicating the result obtained from the user's motion contained in the user information as the motion information indicating the user's motion.

The analysis unit 104 acquires the motion information corresponding to the model of the motion type, for example by reading out the motion information corresponding to the decided model of the motion type from a recording medium such as a storage unit (not shown).

The analysis unit 104 analyzes the user's motion by calculating the difference in motion between the user's motion and the motion of the model of the motion type.

The analysis unit 104 calculates the difference in motion between the user's motion and the motion of the model of the motion type, for example by statistically analyzing a movement indicated by the motion information.

As one example, the analysis unit 104 calculates the difference between motions, for example by analyzing main components of the motion and calculating the difference between main components.

### <III> Notification Processing Unit 106

The notification processing unit 106 plays a role in performing another process using the result obtained from the decision process, and plays a leading role in the notification process as a process using the result obtained from the decision process.

The notification processing unit 106 performs any one process of items (i) to (iv) below as the notification process.

### (i) First Example of Notification Process

The notification processing unit 106 causes a motion indicated by the motion information corresponding to the model of the motion type that is decided in the decision processing unit 102 to be notified.

For example, when the motion information corresponding to the model of the motion type is a captured image obtained by photographing a motion of the model of the motion type, the notification processing unit 106 reads out image data corresponding to the model of the motion type from a recording medium such as a storage unit (not shown). The notification processing unit 106 causes the motion indicated by the motion information corresponding to the model of the motion type to be notified in a visual representation by displaying the captured image indicated by the read image data on a display screen of a display unit (not shown) or a display screen of an external display device.

The notification processing unit 106 is also capable of causing the motion indicated by the motion information corresponding to the model of the motion type to be notified in a visual representation using any representation that can be used to represent a motion such as a stick picture (irrespective of whether the motion information is a captured image).

Furthermore, when the motion information corresponding to the model of the motion type contains audio data or when there is audio data associated with the motion information corresponding to the model of the motion type, the notification processing unit 106 can cause audio indicated by the audio data to be outputted from an audio output device such as a speaker. In this connection, the output of audio indicated by the audio data from an audio output device such as a speaker corresponds to an example of auditory notification of the motion indicated by the motion information corresponding to the model of the motion type.

### (ii) Second Example of Notification Process

The notification processing unit 106 causes the model of the motion type that is decided in the decision processing unit 102 to be notified.

The notification processing unit 106 notifies the decided model of the motion type in a visual representation, for example by causing any contents relating to the model of the motion type such as a name or profile of the model of the motion type to be displayed on a display screen. The notification processing unit 106 may notify the decided model of the motion type in an auditory representation, for example by causing audio indicating any contents relating to the model of the motion type such as a name or profile of the model of the motion type to be outputted from an audio output device such as a speaker.

The notification of the decided model of the motion type allows the user who receives the notification to know a person serving as the model of the motion type for the user.

Thus, it is possible for the user who receives a notification to achieve the skill improvement more efficiently, by “consciously viewing a playing style of a person serving as the model through television broadcast or the like (e.g., when a person serving as the model is a professional sports player)”, by “receiving guidance from a person serving as the model (e.g., when a person serving as the model is a coach in sports or the like)”, by “reading a book in which a motion of a person serving as the model is disclosed (e.g., when a person serving as the model is a professional sports player or a coach in sports or the like)”, or the like.

(iii) Third Example of Notification Process

When the analysis on the user's motion is performed in the analysis unit 104, the notification processing unit 106 causes a result obtained by analyzing the user's motion to be notified.

The notification processing unit 106 causes a result obtained by analyzing the user's motion to be notified in a visual representation, for example by “allowing a difference between motions to be highlighted when one or both of motion information corresponding to the user and motion information corresponding to the model of the motion type are notified in visual representation as described above”. Examples of a method of highlighting the difference between the movements herein include any method capable of highlighting it as a visual representation such as “a method of changing the color of a part having large difference between motions in a captured image, stick picture, or the like” or “a method of blinking a part having large difference between motions in a captured image, stick picture, or the like”, as an example.

### (iv) Fourth Example of Notification Process

The notification processing unit 106 is also capable of performing a process obtained by combining two or more processes of the notification process according to the first example described in the above item (i) to the notification process according to the third example described in the above item (iii).

The information processing apparatus 100 having the configuration shown in FIG. 1 as an example performs the processes for implementing the information processing method according to the present embodiment (e.g., the decision process, the analysis process, and the notification process).

The configuration of the information processing apparatus according to the present embodiment is not limited to that shown in FIG. 1.

For example, the information processing apparatus according to the present embodiment may have a configuration that does not include one or both of the analysis unit 104 and the notification processing unit 106 shown in FIG. 1.

Even when the information processing apparatus according to the present embodiment has a configuration that does not include one or both of the analysis unit 104 and the notification processing unit 106, the information processing apparatus is capable of performing the decision process.

Thus, even when the information processing apparatus according to the present embodiment has a configuration that does not include one or both of the analysis unit 104 and the notification processing unit 106, the information processing apparatus is capable of deciding a model of the motion type that can be used to perform a motion serving as a model for the user. In addition, even when the information processing apparatus according to the present embodiment has a configuration that does not include one or both of the analysis unit 104 and the notification processing unit 106, the information processing apparatus can achieve the effects achieved from the decision process described above.

When the information processing apparatus according to the present embodiment has a configuration that does not include the analysis unit 104, the analysis process may be performed in an external apparatus having a function similar to that of the analysis unit 104. When the information processing apparatus according to the present embodiment has a configuration that does not include the notification processing unit 106, the notification process may be performed in an external apparatus having a function similar to that of the notification processing unit 106. In other words, “the decision process, and one or both of the analysis process and the notification process” for implementing the information processing method according to the present embodiment may be performed, for example, by the information processing apparatus according to the present embodiment and an information processing system including one or more external apparatuses.

As described above, “the decision process”, “the decision process, and one or both of the analysis process and the notification process” are those obtained by dividing the process for implementing the information processing method according to the present embodiment for convenience sake. Thus, the components used to perform the process for implementing the information processing method according to the present embodiment are not limited to the decision processing unit 102, the analysis unit 104, and the notification processing unit 106, shown in FIG. 1, but can have a configuration depending on a way of dividing the process for implementing the information processing method according to the present embodiment.

### <VI> Example of Process for Implementing Information Processing Method According to Present Embodiment

An example of the process for implanting the information processing method according to the present embodiment in the information processing apparatus 100 shown in FIG. 1 will be described.

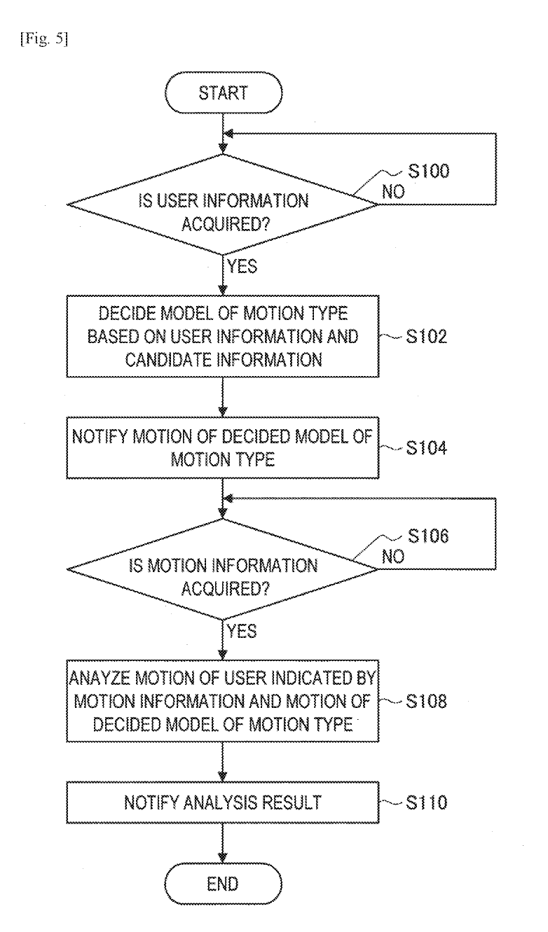

FIG. 5 is a flowchart illustrating an example of the process for implementing the information processing method according to the present embodiment, and illustrates an example of the process in the information processing apparatus 100 shown in FIG. 1. In FIG. 5, steps S100 and S102 correspond to the decision process. In FIG. 5, step S104 corresponds to an example of the notification process, and step S110 corresponds to another example of the notification process. In FIG. 5, steps S106 and S108 s correspond to the analysis process.

In the information processing apparatus 100 shown in FIG. 1, the process in steps S100 and S102 is performed, for example, by the decision processing unit 102. In the information processing apparatus 100 shown in FIG. 1, the process in steps S104 and S110 is performed, for example, by the notification processing unit 106, and the process in steps S106 and S108 is performed, for example, by the analysis unit 104.

The information processing apparatus 100 determines whether the user information is acquired (S100). The user information is acquired, for example by the information processing apparatus 100 reading out it from a recording medium or the like, or by the information processing apparatus 100 acquiring the user information transmitted from an external apparatus.

If it is not determined in step S100 that the user information is acquired, the information processing apparatus 100 does not proceed to the next step until it is determined that the user information is acquired in step S100.

On the other hand, if it is determined in step S100 that the user information is acquired, the information processing apparatus 100 decides a model of the motion type on the basis of the user information and candidate information (S102). For example, the information processing apparatus 100 decides a model of the motion type, for example by performing any one of the process according to the first example described in the above item (1) to the process according to the fourth example described in the above item (4).

When the model of the motion type is decided in step S102, the information processing apparatus 100 notifies a motion of the decided model of the motion type (S104). The information processing apparatus 100 notifies a motion of the decided model of the motion type, for example by performing the notification process according to the first example described in the above item (1).

The information processing apparatus 100 determines whether the motion information corresponding to the user is acquired (S106). The motion information corresponding to the user is acquired, for example by the information processing apparatus 100 reading out it from a recording medium or the like, or by the information processing apparatus 100 acquiring the motion information transmitted from an external apparatus. In addition, when the acquired user information contains the motion information as described above, the information processing apparatus 100 allows the motion information contained in the user information to be used in performing the process.

If it is not determined in step S106 that the motion information corresponding to the user is acquired, the information processing apparatus 100 does not proceed to the next step until it is determined that the motion information is acquired in step S106.

If it is determined in step S106 that the motion information corresponding to the user is acquired, the information processing apparatus 100 analyzes the user's motion indicated by the motion information corresponding to the user and the motion of the model of the motion type decided in step S102 (S108). The information processing apparatus 100 analyzes the user's motion and the motion of the model of the motion type, for example by calculating the difference in coefficients of main components between the user's motion and the motion of the model of the motion type, using any technique such as analysis of main components of the motion.

When the user's motion and the motion of the model of the motion type are analyzed in step S108, the information processing apparatus 100 notifies an analysis result obtained by the analysis (S110). The information processing apparatus 100 notifies the analysis result, for example by performing the notification process according to the third example described in the above item (iii).

The information processing apparatus 100 performs, for example, the process shown in FIG. 5 as the process for implementing the information processing method according to the present embodiment.

The process for implementing the information processing method according to the present embodiment is not limited to the process shown in FIG. 5.

For example, the information processing apparatus 100 can be configured not to perform one or both of the process in step S104 and the process in steps S106 to S110. The information processing apparatus 100 can be configured not to perform the process in step S110, as an example.

Even when one or both of the process in step S104 and the process in steps S106 to S110 are not performed (or when the process in step S110 is not performed), the information processing apparatus 100 performs the decision process.

Thus, even when one or both of the process in step S104 and the process in steps S106 to S110 are not performed (or alternatively when the process in step S110 is not performed), the information processing apparatus according to the present embodiment is capable of deciding a model of the motion type that can be used to perform the motion serving as the model for the user. In addition, even when one or both of the process in step S104 and the process in steps S106 to S110 are not performed (or alternatively when the process in step S110 is not performed), the information processing apparatus according to the present embodiment can achieve the effects achieved from the decision process described above. When the information processing apparatus 100 is configured not to perform one or both of the process in step S104 and the process in steps S106 to S110 are not performed (or alternatively when the process in step S110 is not performed), such process, which is not performed, may be performed by an external apparatus of the information processing apparatus 100.

### <V> Example of Effects Achieved by Using Information Processing Method According to Present Embodiment

The information processing apparatus 100 can achieve the effects described below as an example. It will be understood that the effects achieved by using the information processing method according to the present embodiment are not limited to those described below.
Search for a professional sports player having the most analogous feature of the body or the motion can be performed for each person and a motion serving as the model can be presented for each individual.
Difference between the motion of the professional player serving as the model and the motion of the individual can be presented.
Such presentation described above allows each individual to achieve the improvement of a skill such as sports more efficiently.

As one specific example, the presentation as described above allows a junior doctor (an example of the user) to imitate the movement of an experienced doctor (an example of the model of the motion type) serving as the model for the junior doctor, thereby improving the skill of the procedure in the medical practice, such as surgery.

### <VI> Application Example of Information Processing Apparatus According to Present Embodiment

In the above, the information processing apparatus has been described as one example, but embodiments of the present disclosure are not limited thereto. The present embodiment is applicable to various types of devices capable of performing the process for implementing the information processing method according to the present embodiment. Examples of such devices include a computer such as personal computers (PCs) and servers, tablet type apparatus, communication apparatus such as mobile phones and smartphones, wearable device used by the user while being worn. The present embodiment is applicable to a processing IC that can be incorporated into such devices.

The information processing apparatus according to the present embodiment may be applied to a system including a plurality of devices under the condition that the devices are connected to a network (or communication between apparatuses), such as cloud computing. In other words, the information processing apparatus according to the present embodiment described above is also capable of being implemented as an information processing system having a plurality of devices that perform the process for implementing the information processing method according to the present embodiment. An example of the information processing system performing the process for implementing the information processing method according to the present embodiment using the plurality of devices includes a system in which “the decision process”, or “the decision process and one or both of the analysis process and the notification process” is performed in conjunction with a plurality of devices that constitute the information processing system.

### (Program According to Present Embodiment)

A program for causing a computer to function as the information processing apparatus according to the present embodiment may be executed by a processor or the like in the computer (e.g., program capable of executing the process for implementing the information processing method according to the present embodiment, such as “the decision process”, “the decision process, and one or both of the analysis process and the notification process”), and thus it is possible to decide a model of the motion type that can be used to perform a motion serving as the model for the user.

Moreover, when a program that causes a computer to function as the information processing apparatus according to the present embodiment is executed by a processor or the like in the computer, it is possible to provide an effect provided by the processing related to the information processing method according to the present embodiment described above.

It should be understood by those skilled in the art that various modifications, combinations, sub-combinations and alterations may occur depending on design requirements and other factors insofar as they are within the scope of the appended claims or the equivalents thereof.

For example, it has been illustrated above that a program (computer program) that causes a computer to function as the information processing apparatus according to the present embodiment is provided, but the present embodiment can further provide a recording medium in which the above-described program is stored together.

The above-described configurations express examples of the embodiment and, of course, pertain to the technical scope of the present disclosure.

In addition, the effects described in the present specification are merely illustrative and demonstrative, and not limitative. In other words, the technology according to the present disclosure can exhibit other effects that are evident to those skilled in the art along with or instead of the effects based on the present specification.

Additionally, the present technology may also be configured as below.

(1)

An information processing method, the method comprising:

comparing user information to a plurality of data sets, each data set of the plurality of data sets has candidate information including a candidate model of a motion type for a user; and
selecting one candidate model of one data set of the plurality of data sets as a selected model of the motion type for the user based on the comparison of the user information to the plurality of data sets.
(2)

The information processing method according to (1),

wherein the user information is indicative of a user feature of a user body,
wherein the candidate information is indicative a candidate feature of a candidate body,
wherein comparing the user information the plurality of data sets further includes comparing the user feature of the user body to the candidate feature of the candidate body, and
wherein selecting the one candidate model of the one data set of the plurality of data sets as the selected model of the motion type for the user is based on the candidate feature of the candidate body that is most similar to the user feature of the user body.
(3)

The information processing method according to (2),

wherein the user information is indicative of one or more user feature values indicating the user feature of the user body,
wherein the candidate information is indicative of one or more candidate feature values indicating the candidate feature of the candidate body,
wherein comparing the user information the plurality of data sets further includes comparing the one or more user feature values indicating the user feature of the user body to the one or more candidate feature values indicating the candidate feature of the candidate body, and
wherein selecting the one candidate model of the one data set of the plurality of data sets as the selected model of the motion type for the user is based on a sum total value of a difference between the one or more user feature values indicating the user feature of the user body and corresponding the one or more candidate feature values indicating the candidate feature of the candidate body.
(4)

The information processing method according to (3), further comprising

classifying the one or more user feature values of the user information and corresponding the one or more candidate feature values of the candidate information into a plurality of segments: and
weighting the plurality of segments.
(5)

The information processing method according to (3), wherein selecting the one candidate model of the one data set of the plurality of data sets as the selected model of the motion type for the user further includes selecting the one candidate model of the one data set of the plurality of data sets with a smallest sum total value.

(6)

The information processing method according to any of (1) through (5),

wherein the user information is indicative of a first result obtained from a first motion,
wherein the candidate information is indicative of a second result obtained from a second motion,
wherein comparing the user information the plurality of data sets further includes comparing the first result to the second result, and
wherein selecting the one candidate model of the one data set of the plurality of data sets as the selected model of the motion type for the user further includes selecting the one candidate model of the one data set of the plurality of data sets based on a difference between the first result and the second result.
(7)

The information processing method according to (6), further comprising:

extracting a first feature of the first motion from the user information; and
extracting a second feature of the second motion from the candidate information,
wherein comparing the user information to the plurality of data sets further includes comparing the first feature to the second feature extracted from the each data set of the plurality of data sets, and
wherein selecting the one candidate model of the one data set of the plurality of data sets as the selected model of the motion type for the user is based on a difference between the first feature and the second feature extracted from the each data set of the plurality of data sets.
(8)

The information processing method according to (7),

wherein extracting the first feature of the first motion of the user information includes extracting a user posture from the user information,
wherein extracting the second feature of the second motion from the candidate information includes extracting a candidate posture from the candidate information,
wherein comparing the user information to the plurality of data sets further includes comparing the user posture to the candidate posture extracted from the each data set of the plurality of data sets, and
wherein selecting the one candidate model of the one data set of the plurality of data sets as the selected model of the motion type for the user is based on a smallest difference between the user posture and the candidate posture extracted from the each data set of the plurality of data sets.
(9)

The information processing method according to (6) or (7), further comprising:

calculating a first feature value of the first motion from the user information,
calculating a second feature value of the second motion from the candidate information,
wherein comparing the user information to the plurality of data sets further includes comparing the first feature value to the second feature value calculated from the each data set of the plurality of data sets, and
wherein selecting the one candidate model of the one data set of the plurality of data sets as the selected model of the motion type for the user is based on a smallest difference between the first feature value and the second feature value calculated from the each data set of the plurality of data sets.
(10)

The information processing method according to any of (1) through (9),

wherein the user information is indicative of a user gender,
wherein the candidate information is indicative of a candidate gender,
wherein comparing the user information to the plurality of data sets further includes comparing the user gender to the candidate gender in each of the plurality of data sets, and
wherein selecting the one candidate model of the one data set of the plurality of data sets as the selected model of the motion type for the user further includes excluding the each data of the plurality of data sets with the candidate gender that is different than the user gender.
(11)

The information processing method according to any of (1) through (10),

wherein the user information is indicative of a user age,
wherein the candidate information is indicative of a candidate age,
wherein comparing the user information to the plurality of data sets further includes comparing the user age to the candidate age in each of the plurality of data sets, and
wherein selecting the one candidate model of the one data set of the plurality of data sets as the selected model of the motion type for the user further includes excluding the each data set of the plurality of data sets with the candidate age that has an age gap from the user age that is greater than or equal to a predetermined threshold.
(12)

The information processing method according to any of (1) through (11), further comprising notifying a motion indicated by motion information from the one candidate model selected as the selected model of the motion type.

(13)

The information processing method according to any of (1) through (12), further comprising analyzing a motion of the user based on first motion information indicating the motion of the user and corresponding second motion information from the one candidate model selected as the selected model of the motion type.

(14)

The information processing method according to (13), wherein analyzing the motion of the user further includes calculating a difference between the first motion information indicating the motion of the user and the corresponding second motion information from the one candidate model selected as the selected model of the motion type.

(15)

The information processing method according to (13) or (14), further comprising notifying a result obtained by the analysis of the motion of the user.

(16)

The information processing method according to any of (1) through (15), wherein the one candidate model selected as the model of the motion type for the user is a model of a sports player.

(17)

The information processing method according to any of (1) through (15), wherein the one candidate model selected as the model of the motion type for the user is a model of a medical professional.

(18)

The information processing method according to any of (1) through (15), wherein the one candidate model selected as the model of the motion type for the user is a model of a user that operates with a specific motion.

(19)

An information processing apparatus comprising:

an electronic processor; and
a memory storing instructions that, when executed by the electronic processor, perform a set of operations including
comparing user information to a plurality of data sets, each data set of the plurality of data sets has candidate information including a candidate model of a motion type for a user, and
selecting one candidate model of one data set of the plurality of data sets as a selected model of the motion type for the user based on the comparison of the user information to the plurality of data sets.
(20)

An information processing system comprising:

a plurality of devices connected to a network, wherein one or more of the plurality of devices is configured to
compare user information to a plurality of data sets, each data set of the plurality of data sets has candidate information including a candidate model of a motion type for a user, and
select one candidate model of one data set of the plurality of data sets as a selected model of the motion type for the user based on the comparison of the user information to the plurality of data sets.

### REFERENCE SIGNS LIST

100 information processing apparatus
102 decision processing unit
104 analysis unit
106 notification processing unit

1. An information processing method, the method comprising:
comparing user information to a plurality of data sets, each data set of the plurality of data sets has candidate information including a candidate model of a motion type for a user; and
selecting one candidate model of one data set of the plurality of data sets as a selected model of the motion type for the user based on the comparison of the user information to the plurality of data sets.

2. The information processing method according to claim 1,
wherein the user information is indicative of a user feature of a user body,
wherein the candidate information is indicative a candidate feature of a candidate body,
wherein comparing the user information the plurality of data sets further includes comparing the user feature of the user body to the candidate feature of the candidate body, and
wherein selecting the one candidate model of the one data set of the plurality of data sets as the selected model of the motion type for the user is based on the candidate feature of the candidate body that is most similar to the user feature of the user body.

3. The information processing method according to claim 2,
wherein the user information is indicative of one or more user feature values indicating the user feature of the user body,
wherein the candidate information is indicative of one or more candidate feature values indicating the candidate feature of the candidate body,
wherein comparing the user information the plurality of data sets further includes comparing the one or more user feature values indicating the user feature of the user body to the one or more candidate feature values indicating the candidate feature of the candidate body, and
wherein selecting the one candidate model of the one data set of the plurality of data sets as the selected model of the motion type for the user is based on a sum total value of a difference between the one or more user feature values indicating the user feature of the user body and corresponding the one or more candidate feature values indicating the candidate feature of the candidate body.

4. The information processing method according to claim 3, further comprising
classifying the one or more user feature values of the user information and corresponding the one or more candidate feature values of the candidate information into a plurality of segments; and
weighting the plurality of segments.

5. The information processing method according to claim 3,
wherein selecting the one candidate model of the one data set of the plurality of data sets as the selected model of the motion type for the user further includes selecting the one candidate model of the one data set of the plurality of data sets with a smallest sum total value.

6. The information processing method according to claim 1,
wherein the user information is indicative of a first result obtained from a first motion,
wherein the candidate information is indicative of a second result obtained from a second motion,
wherein comparing the user information the plurality of data sets further includes comparing the first result to the second result, and
wherein selecting the one candidate model of the one data set of the plurality of data sets as the selected model of the motion type for the user further includes selecting the one candidate model of the one data set of the plurality of data sets based on a difference between the first result and the second result.

7. The information processing method according to claim 6, further comprising:
extracting a first feature of the first motion from the user information, and
extracting a second feature of the second motion from the candidate information,
wherein comparing the user information to the plurality of data sets further includes comparing the first feature to the second feature extracted from the each data set of the plurality of data sets, and
wherein selecting the one candidate model of the one data set of the plurality of data sets as the selected model of the motion type for the user is based on a difference between the first feature and the second feature extracted from the each data set of the plurality of data sets.

8. The information processing method according to claim 7,
wherein extracting the first feature of the first motion of the user information includes extracting a user posture from the user information,
wherein extracting the second feature of the second motion from the candidate information includes extracting a candidate posture from the candidate information,
wherein comparing the user information to the plurality of data sets further includes comparing the user posture to the candidate posture extracted from the each data set of the plurality of data sets, and
wherein selecting the one candidate model of the one data set of the plurality of data sets as the selected model of the motion type for the user is based on a smallest difference between the user posture and the candidate posture extracted from the each data set of the plurality of data sets.

9. The information processing method according to claim 6, further comprising:
calculating a first feature value of the first motion from the user information,
calculating a second feature value of the second motion from the candidate information,
wherein comparing the user information to the plurality of data sets further includes comparing the first feature value to the second feature value calculated from the each data set of the plurality of data sets, and
wherein selecting the one candidate model of the one data set of the plurality of data sets as the selected model of the motion type for the user is based on a smallest difference between the first feature value and the second feature value calculated from the each data set of the plurality of data sets.

10. The information processing method according to claim 1,
wherein the user information is indicative of a user gender,
wherein the candidate information is indicative of a candidate gender,
wherein comparing the user information to the plurality of data sets further includes comparing the user gender to the candidate gender in each of the plurality of data sets, and
wherein selecting the one candidate model of the one data set of the plurality of data sets as the selected model of the motion type for the user further includes excluding the each data of the plurality of data sets with the candidate gender that is different than the user gender.

11. The information processing method according to claim 1,
wherein the user information is indicative of a user age,
wherein the candidate information is indicative of a candidate age,
wherein comparing the user information to the plurality of data sets further includes comparing the user age to the candidate age in each of the plurality of data sets, and
wherein selecting the one candidate model of the one data set of the plurality of data sets as the selected model of the motion type for the user further includes excluding the each data set of the plurality of data sets with the candidate age that has an age gap from the user age that is greater than or equal to a predetermined threshold.

12. The information processing method according to claim 1, further comprising notifying a motion indicated by motion information from the one candidate model selected as the selected model of the motion type.

13. The information processing method according to claim 1, further comprising analyzing a motion of the user based on first motion information indicating the motion of the user and corresponding second motion information from the one candidate model selected as the selected model of the motion type.

14. The information processing method according to claim 13,
wherein analyzing the motion of the user further includes calculating a difference between the first motion information indicating the motion of the user and the corresponding second motion information from the one candidate model selected as the selected model of the motion type.

15. The information processing method according to claim 13, further comprising notifying a result obtained by the analysis of the motion of the user.

16. The information processing method according to claim 1,
wherein the one candidate model selected as the model of the motion type for the user is a model of a sports player.

17. The information processing method according to claim 1,
wherein the one candidate model selected as the model of the motion type for the user is a model of a medical professional.

18. The information processing method according to claim 1,
wherein the one candidate model selected as the model of the motion type for the user is a model of a user that operates with a specific motion.

19. An information processing apparatus comprising:
an electronic processor; and
a memory storing instructions that, when executed by the electronic processor, perform a set of operations including
comparing user information to a plurality of data sets, each data set of the plurality of data sets has candidate information including a candidate model of a motion type for a user, and
selecting one candidate model of one data set of the plurality of data sets as a selected model of the motion type for the user based on the comparison of the user information to the plurality of data sets.

20. An information processing system comprising:
a plurality of devices connected to a network, wherein one or more of the plurality of devices is configured to
compare user information to a plurality of data sets, each data set of the plurality of data sets has candidate information including a candidate model of a motion type for a user, and
select one candidate model of one data set of the plurality of data sets as a selected model of the motion type for the user based on the comparison of the user information to the plurality of data sets.
